(19) United States
(12) Patent Application Publication
(10) Pub. No.: US 2008/0177292 A1
(43) Pub. Date: Jul. 24, 2008

(54) Vertically oriented band for stomach

(76) Inventors: Moises Jacobs, Miami, FL (US); Moises Jacobs, Miami, FL (US)

Correspondence Address: Christopher J. McDonald;HOFFMAN, WASSON & GITLER, P.C., Suite 522, 2461 South Clark Street, Arlington, VA, 22202, US

(21) Appl. No.: 11/797,537

(22) Filed: May 4, 2007

Related U.S. Application Data
(60) Provisional application No. 60/881,138, filed on Jan. 19, 2007.

(51) Int. Cl. A61M 29/00

(52) U.S. Cl. 606/157

(57) ABSTRACT

A laparoscopic band or non-adjustable clamp is placed about the greater curvature of the stomach in a vertical orientation. The band or clamp completely compartmentalizes the stomach between a small pouch and the fundus. The fundic part of the stomach is excluded from nutrients and creates a long narrow channel where the food travels. A small passage at the level of the antrum allows gastric juices to empty from the fundic areas.

This application claims benefit of provisional application 60/881,138 filed Jan. 19, 2007.

### BACKGROUND OF THE INVENTION

For patients whose obesity presents an immediate serious health risk, surgical procedures are available to promote weight loss. One of the most common surgical procedures is gastric bypass. During gastric bypass, the stomach is made smaller and food bypasses part of the small intestine. The smaller size stomach causes the patient to eat less before the stomach is full and the bypass of the small intestines leads to less calories being absorbed by the body.

In the most common type of gastric bypass surgery, roux-en-y, a small pouch is formed at the top of the stomach using staples or a plastic band. The smaller stomach is connected to the middle portion of the small intestines bypassing the upper portion of the small intestines.

Devices have been developed to form the smaller stomach from the patient's original stomach. One such device is disclosed in U.S. 2002/0022851 (Kalloo et al). The Kalloo et al patent discloses a loop 80 reducing the volume of the gastric cavity. A feeder line is pulled to reduce the diameter of the loop and collapse the walls of the stomach to define a smaller pouch.

Saadat et al (2006/0157067) discloses the use of tissue anchors to form a gastric pouch acting as a restriction to the passage of fluids and food. U.S. Pat. No. 5,345,949 (Shlain) discloses a clip placed across the fundus of the stomach to restrict the inlet chamber or proximal pouch. Likewise, U.S. Pat. No. 6,869,438 (Chao) discloses a gastric partitioning clip creating a stomach pouch from the stomach to restrict the amount of food intake.

It is an object of the invention to provide a device for separating the stomach into two compartments but allowing communication between the compartments.

It is another object of the invention to provide a device for forming a smaller stomach pouch, the size of the pouch being tailored to the patient's individual circumstances.

It is another object of the invention to provide a procedure creating a small stomach pouch to limit intake of food separate from the stomach but allowing gastric juices from the stomach to flow into the pouch.

It is still another object of the invention to provide a system for creating a small pouch from the main stomach that is reversible.

It is still another object of the invention to alter the production of hormones, enzymes and chemicals that affect metabolism, energy levels, hunger, digestion, absorption of nutrients that may be affected by exclusion of the gastric fundus.

These and other objects of the invention will become apparent after reading the disclosure of the invention.

### SUMMARY OF THE INVENTION

A laparoscopic adjustable band or an adjustable or non-adjustable clamp is placed about the greater curvature of the stomach in a vertical orientation. The band or clamp completely compartmentalizes the stomach between a small vertical pouch and the fundus. The fundic part of the stomach is excluded from nutrients and is separated from a long narrow channel where the food travels. A small passage at the level of the antrum allows gastric juices to empty from the fundic areas. The band may be applied during open surgery or through a trochar.

### BRIEF DESCRIPTION OF THE DRAWINGS

FIG. 1 is a view of the band applied to a stomach;

FIG. 2 is a cross-sectional view of an inflatable band useable with the invention;

FIG. 3 is a view of a non-adjustable clamp used with the invention;

FIG. 4 is a detailed view of the strap attachment to the band;

FIG. 5 is a perspective view of a second embodiment of the band; and

FIG. 6 is a view of the band in FIG. 5 applied to a stomach.

### DETAILED DESCRIPTION OF THE INVENTION

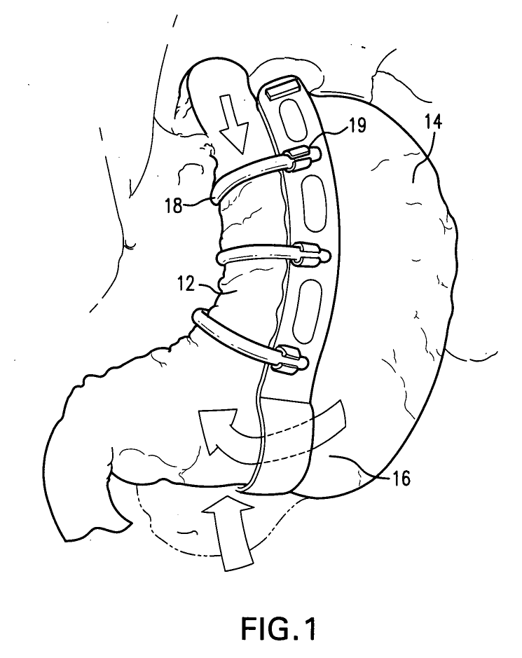

In FIG. 1, a stomach having the band 10 applied can be seen dividing the stomach into the pouch 12 and fundic area 16. Food traveling down the esophagus enters the pouch 12 and exits into the antrum. The band 10 applies pressure against the sides of the stomach to separate the stomach into the two compartments 12, 14 but does not apply pressure to the stomach walls at the bottom part of the stomach. This creates a passage 16 allowing flow of gastric juices from the fundic area 14 into the antrum. Food will not enter the fundic area through this passage, however. At least one horizontal strap 18 may be used to secure the band in place. The straps do not apply pressure sufficient to impact the size and function of the compartment 12.

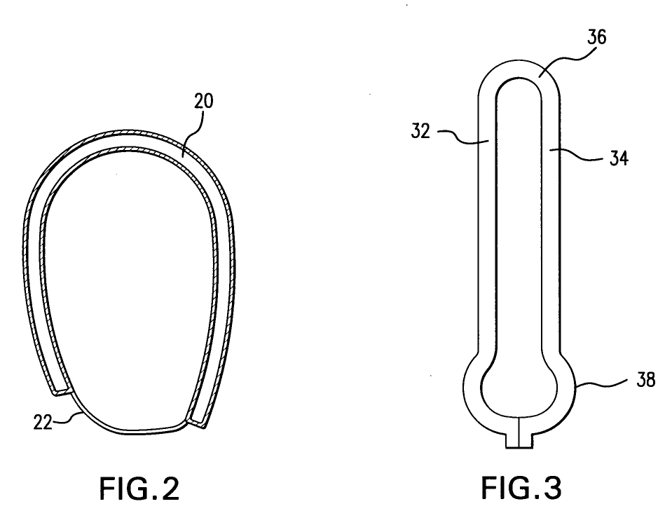

FIG. 2 shows an embodiment of the band having an inflatable chamber 20 and a connecting section 22. The band is placed about the stomach in a vertical orientation to separate the stomach into the two compartments and inflated. The inflated chamber 20 applies pressure on the stomach to seal the two compartments from one another except for the passage 16. The connecting section 22, being not inflated, does not apply pressure to the bottom portion of the stomach, allowing for the formation of the passage 16.

FIG. 3 shows the rigid clamp embodiment having a U portion formed by two legs 32, 34 connected by a bight portion 36. When the clamp is placed on the stomach, the bight portion 36 fits over the top of the stomach with the legs 32, 34 applying enough pressure to collapse the walls of the stomach against one another to create the two compartments. The legs 32, 34 do not extend the full vertical extent of the stomach to allow for the creation of the passage 16. The legs are attached by a connector 38. When applied to the stomach, the legs serves to push the sides of the stomach together to form a complete seal but the connector allows for the formation of a passage between the two compartments 12, 16. The clamp may be adjustable. The legs of the clamp may be made of any length so that depending on the patient's condition and prior uses of the band, the legs can be made shorter or longer and the band moved to the right, forming a larger pouch 12 and a smaller fundic portion 14.

Besides a clip, the vertical band may be form as or with an inflatable balloon, as discussed with reference to FIG. 2. The orientation of the balloon is such that, upon inflation, the balloon bulges to the left to decrease the size of the compartment 12. The bottom portion may or may not be inflatable. The balloon may be attached to a tube exiting the body so that the balloon may be adjusted without the need for invasive surgery.

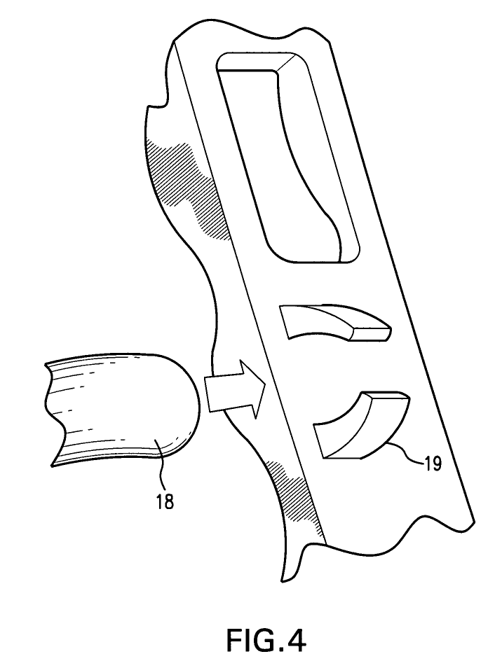

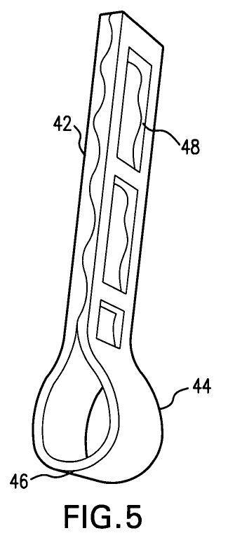

Straps 18 can be secured to the band in any number of conventional ways. One possible way in which to secure the straps to the band is depicted in FIG. 4. The band 18 engages and is secured by clips 19 which extend outwardly from the band. This arrangement allows the straps to be tightened by being pulled through the clip and, if desired, the straps can be released for the removal of the band.

An alternative construction of the band is seen in FIG. 5. In this embodiment, the band has a first section 42 having two parallel arms and a second section with two space members so that, when applied to a stomach, the passageway 16 is formed. The arms may be straight, curved or undulating. The surface may be smooth or serrated. The arms of the first section 42 are resiliently biased against one another and are spaced from one another in order that, when applied, the first section maintains the walls of the stomach together to separate the stomach into the first and second compartments 12, 14. The pressure applied must be enough that the two compartments are formed but not so much that the walls of the stomach are damaged or compromise the blood supply. The section 44 is connected together by a section 46 acting as a hinge. This allows the arms of the first section 42 to be separated from one another in order that the band may be applied. Conversely, it is possible to have the two arms of the first section 42 hinged to one another and the two arcuate portions forming the second section 44 not connected to one another.

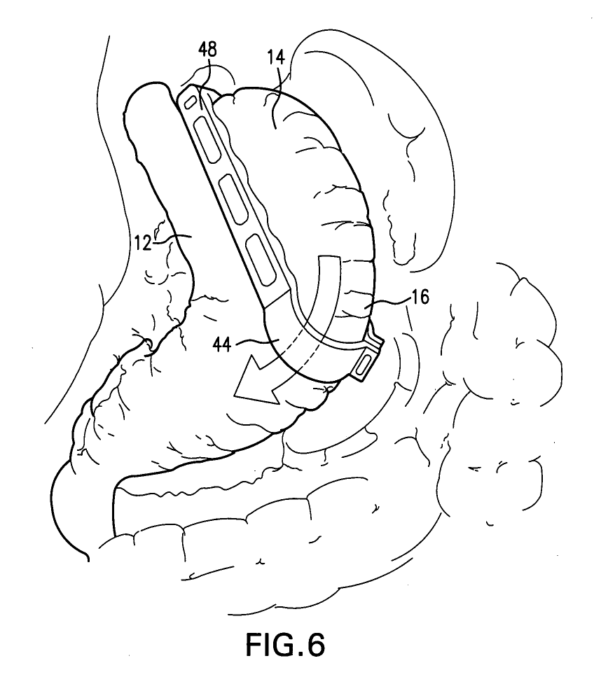

The band of FIG. 5 applied to stomach is seen in FIG. 6. Seen here as the first section 42 extending along the stomach to separate the stomach to separate the stomach into two compartments 12, 14 whereas the second section has arcuate arms forming a passage 16. At least one of the arms of the first section is provided with apertures 48. The apertures, which may be large or small, allow part of the stomach wall to enter the aperture to help prevent movement of the band once it has been applied.

While the invention has been described with reference to preferred embodiments, various modifications would be apparent to one of ordinary skill in the art. The invention encompasses such variations and modifications.

What is claimed is:

1. A gastric band, comprising:
a first section having two arms spaced apart by a first distance; and
a second section having two arms spaced apart by a second distance;
the first distance being smaller than the second distance.

2. The gastric band of claim 1, wherein the arms of the first section are parallel and the arms of the second section are arcuate.

3. The gastric band of claim 1, further comprising apertures in at least one of the first section arms.

4. The gastric band of claim 1, wherein the first section and second section form a closed loop.

5. The gastric band of claim 1, wherein the first section and second section form a U-shape.

6. The gastric band of claim 1, wherein the second section two arms are hinged to one another.

7. A gastric band, comprising
a first section having two arms, the two arms spaced from one another a distance to close the walls of a stomach together and form a first and second compartment,
a second section having two arms, the two arms spaced from one another to form a passage between the first and second compartment.

8. The gastric band of claim 7, wherein, the two arms of the first section are inflatable.
